(19) United States
(12) Patent Application Publication
(10) Pub. No.: US 2022/0313715 A1
(43) Pub. Date: Oct. 6, 2022

(54) PHARMACEUTICAL COMPOSITION, FOR PREVENTING OR TREATING HEPATIC FIBROSIS, COMPRISING 8-OHDG

(71) Applicant: GIL MEDICAL CENTER, Incheon (KR)

(72) Inventors: Seung Kak SHIN, Seoul (KR); Oh Sang KWON, Gyeonggi-do (KR); Myung Hee CHUNG, Incheon (KR)

(21) Appl. No.: 16/306,183

(22) PCT Filed: May 29, 2017
(86) PCT No.: PCT/KR2017/005574

§ 371 Date: Apr. 27, 2021

(30) Foreign Application Priority Data
Jun. 2, 2016 (KR) 10-2016-0068986

(51) Int. Cl. A61K 31/708; A61P 1/16

(52) U.S. Cl. CPC A61K 31/708; A61P 1/16

(57) ABSTRACT

The present invention relates to a pharmaceutical composition for preventing or treating liver fibrosis or liver cirrhosis, comprising 8-OHdG or a pharmaceutically acceptable salt thereof, a method for preventing or treating liver fibrosis or liver cirrhosis using the same, and a food composition for ameliorating liver fibrosis or liver cirrhosis, comprising 8-hydroxydeoxyguanosine (8-OHdG) or a pharmaceutically acceptable salt thereof. As use of the pharmaceutical composition for preventing or treating liver fibrosis or liver cirrhosis can reduce levels of various markers whose expression level increases due to induced liver fibrosis, the pharmaceutical composition can be widely used in the effective prevention or treatment of liver fibrosis or liver cirrhosis induced thereby.

### TECHNICAL FIELD

The present invention relates to a pharmaceutical composition for preventing or treating liver fibrosis comprising 8-hydroxydeoxyguanosine (OHdG), more specifically to a pharmaceutical composition for preventing or treating liver fibrosis or liver cirrhosis, comprising 8-OHdG or a pharmaceutically acceptable salt thereof, a method for preventing or treating liver fibrosis or liver cirrhosis using the same, and a food composition for ameliorating liver fibrosis or liver cirrhosis, comprising 8-hydroxydeoxyguanosine (8-OHdG) or a pharmaceutically acceptable salt thereof.

### BACKGROUND ART

Fibrosis refers to a condition in which the wound healing process after tissues are damaged by various stresses (inflammation, chemical stimulation, radiation, etc.) cannot be regulated normally. In particular, the mechanism of fibrosis, which is a common pathway of chronic diseases, is very complicated and has not yet been completely clarified.

When the liver is stimulated by various factors such as alcohol, viruses, etc., hepatic stellate cells are activated by various cytokines including transforming growth factor beta (TGF-β) secreted from Kupffer cells. The secreted TGF-β is known not only to promote collagen synthesis to accumulate extracellular matrix and cause liver fibrosis due to continuously accumulated collagen, but also to affect surrounding hepatocytes in addition to the hepatic stellate cells themselves, leading to epithelial to mesenchymal transition (EMT). Since continuous hepatic fibrosis eventually leads to liver cirrhosis, understanding and studying the process of liver fibrosis is the most fundamental step in resolving all of the diseases that can cause liver cirrhosis.

In contrast to liver cirrhosis, liver fibrosis is generally known as being reversible, consisting of thin fibrils, and not involving nodule formation. When the causes of liver damage disappear, normal recovery may be possible; however, if the liver damage continues repeatedly, crosslinking between the extracellular matrix (ECM) increases, leading to irreversible liver cirrhosis with nodules.

Liver fibrosis progresses to cirrhosis. When hepatocellular necrosis occurs for any reason, hepatocyte regeneration and fibrosis take place, and when this process is repeated over a long time, liver cirrhosis occurs, and which leads to disease such as cirrhosis complications, liver cancer, etc., and ultimately to death. In particular, since symptoms are absent in early stages of liver cirrhosis and only appear after considerable progression, studies are actively underway to develop a method for rapidly diagnosing and treating liver fibrosis.

For example, KR Patent No. 1086040 discloses an agent for treating hepatic fibrosis and liver cirrhosis comprising asiatic acid derivatives, a pharmaceutically acceptable salt thereof, or an ester thereof, and KR Patent No. 1135574 discloses a technique of preventing and treating hepatic fibrosis and cirrhosis, pulmonary fibrosis, scleroderma, renal glomerular fibrosis, etc. using derivatives of N-(2,2-disubstituted-2H-chromen-6-yl)thiourea, while KR Patent No. 1326256 discloses a pharmaceutical composition for prevention or treatment of hepatic fibrosis and liver cirrhosis comprising ramalin.

Under such circumstances, the present inventors endeavored to develop a method for more effectively treating liver fibrosis, and as a result found that liver fibrosis can be ameliorated upon administration of 8-OHdG, thereby completing the present invention.

### DISCLOSURE

### Technical Problem

An object of the present invention is to provide a pharmaceutical composition for preventing or treating liver fibrosis or liver cirrhosis, comprising 8-hydroxydeoxyguanosine (8-OHdG) or a pharmaceutically acceptable salt thereof.

Another object of the present invention is to provide a method for preventing or treating liver fibrosis or liver cirrhosis, comprising administering the pharmaceutical composition to a subject.

Still another object of the present invention is to provide a food composition for ameliorating liver fibrosis or liver cirrhosis, comprising 8-hydroxydeoxyguanosine (8-OHdG) or a pharmaceutically acceptable salt thereof.

Still another object of the present invention is to provide use of 8-hydroxydeoxyguanosine (8-OHdG) or a pharmaceutically acceptable salt thereof for the prevention or treatment of liver fibrosis or liver cirrhosis.

### Technical Solution

While conducting various studies to develop a method for more effectively treating liver fibrosis, the present inventors paid attention to 8-OHdG. The 8-OHdG has been used as a biomarker of oxidative stress by measuring 8-OHdG contained in isolated DNA or that removed by a repair enzyme and contained in urea. Recent studies have newly revealed that through competition with GTP, 8-OHdG binds to RAC1-GTP and inhibits RAC1 activation, which leads to inhibition of activity of NOX complex, and this ultimately results in strong anti-oxidative and anti-inflammatory actions. However, the effect of 8-OHdG on liver fibrosis has not yet been reported.

The present inventors constructed an animal model having liver fibrosis through ligation of the common bile duct, and studied changes caused according to 8-OHdG administration. As a result, they found that the levels of various biomarkers of liver fibrosis increased in the liver tissues of the animal model having liver fibrosis, but were reduced by administration of 8-OHdG, thereby newly investigating use of 8-OHdG for treating liver fibrosis. The therapeutic effect of 8-OHdG on liver fibrosis has thus far been unknown, and was first investigated by the present inventors.

In order to achieve the objects, the present invention provides, as an aspect, a pharmaceutical composition for preventing or treating liver fibrosis, comprising 8-hydroxydeoxyguanosine (8-OHdG) or a pharmaceutically acceptable salt thereof.

As used herein, the term “8-hydroxydeoxyguanosine (8-OHdG)” refers to an oxidized derivative compound of deoxyguanosine, which is also called “8-oxo-2′-deoxyguanosine” or “8-oxo-dG”. As 8-OHdG is an oxidation product, its intracellular concentration can be used for quantification of the level of oxidative stress exerted on the cells.

As used herein, the term “pharmaceutically acceptable salt” refers to a salt that can be pharmaceutically used, among the substances having cations and anions coupled by electrostatic attraction. Conventionally, it may include metal salts, salts with organic bases, salts with inorganic acids, salts with organic acids, salts with basic or acidic amino acids, or the like. Examples of the metal salts may include alkali metal salts (sodium salts, potassium salts, etc.), alkaline earth metal salts (calcium salts, magnesium salts, barium salts, etc.), aluminum salts, etc.; examples of the salts with organic bases may include salts with triethylamine, pyridine, picoline, 2,6-lutidine, ethanolamine, diethanolamine, triethanolamine, cyclohexylamine, dicyclohexylamine, N,N′-dibenzylethylenediamine, etc.; examples of the salts with inorganic acids may include salts with hydrochloric acid, hydrobromic acid, nitric acid, sulfuric acid, phosphoric acid, etc.; examples of the salts with organic acids may include salts with formic acid, acetic acid, trifluoroacetic acid, phthalic acid, fumaric acid, oxalic acid, tartaric acid, maleic acid, citric acid, succinic acid, ethanesulfonic acid, benzenesulfonic acid, p-toluenesulfonic acid, etc.; examples of the salts with basic amino acids may include salts with arginine, lysine, ornithine, etc.; and examples of the salt with acidic amino acids include salts with aspartic acid, glutamic acid, etc.

In particular, when the compound comprises an acidic functional group, preferred examples of salts are inorganic salts such as alkali metal salts (e.g., sodium salts, potassium salts), alkali earth metal salts (e.g., calcium salts, magnesium salts, barium salts), and organic salts such as ammonium salts. When the compound comprises a basic functional group, preferred examples of salts are salts with inorganic acids, such as hydrochloric acid, hydrobromic acid, nitric acid, sulfuric acid, and phosphoric acid, and salts with organic acids, such as acetic acid, phthalic acid, fumaric acid, oxalic acid, tartaric acid, maleic acid, citric acid, succinic acid, methanesulfonic acid, and p-toluenesulfonic acid.

As used herein, the term “liver fibrosis” refers to a disease which shows a liver tissue having hepatocytes which have not recovered to normal condition but converted to a fibrous tissue such as collagen. Liver fibrosis is known to mainly appear after necrosis of hepatocytes due to liver damage and after interface hepatitis which causes portal-portal fibrous bridges of zone 1. Specific relevant diseases include liver cirrhosis, etc.

As used herein, the term “liver cirrhosis” refers to a liver disease in which continuous destruction of hepatocytes and repeated diffuse hepatic damage are caused by hepatitis, etc., thereby causing liver fibrogenesis and regeneration nodules. Liver cirrhosis mostly involves liver stiffness and distortion of liver shape, and because the structure of the liver lobules is mostly destroyed, it also involves destruction of the three-dimensional positional relationship between the portal vein and the central vein. It is known that about 20% to 40% of liver cirrhosis progresses to liver cancer. When liver fibrosis progresses to liver cirrhosis, the liver cannot be restored to its normal state, and thus, it can be treated using the pharmaceutical composition of the present invention, which targets the progress of liver fibrosis.

As used herein, the term “prevention” refers to all behaviors resulting in the suppression or delay of the onset of liver fibrosis or liver cirrhosis from administering the pharmaceutical composition for preventing or treating liver fibrosis or liver cirrhosis according to the present invention.

As used herein, the term “treatment” refers to all behaviors resulting in the amelioration of liver fibrosis or liver cirrhosis, conversion of cells developing liver fibrosis into normal cells, and other beneficial alterations of the diseases from administering the pharmaceutical composition for preventing or treating liver fibrosis or liver cirrhosis according to the present invention.

The 8-OHdG provided in the present invention can inhibit the expression of various biomarkers of reactive oxygen species (ROS) formation process and liver fibrosis, such as hydroxyproline, transforming growth factor β (TGF-β), tissue inhibitor of metalloproteinases 1 (TIMP-1), collagen, α-smooth muscle actin (α-SMA), NADPH oxidase 1 (NOX1), NADPH oxidase 2 (NOX2), and ras-related C3 botulinum toxin substrate 1 (Rac1), in a liver tissue.

According to an exemplary embodiment of the present invention, a liver fibrosis animal model was prepared by performing bile duct ligation in rats, and the changes in the expression level of the liver fibrosis biomarker according to the administration of 8-OHdG were compared. As a result, the hydroxyproline level drastically increased in the liver tissue of the liver fibrosis animal model, whereas it decreased upon the administration of 8-OHdG (FIG. 1). The mRNA level of transforming growth factor β (TGF-β), tissue inhibitor of metalloproteinases 1 (TIMP-1), collagen, α-smooth muscle actin (α-SMA), NADPH oxidase 1 (NOX1), and NADPH oxidase 2 (NOX2) in the liver tissue also significantly increased in the liver fibrosis animal model, but decreased after 8-OHdG was administered (FIGS. 2a to 2f). The protein level of collagen, α-SMA, NOX1, and NOX2 in the liver tissue of the liver fibrosis animal model also drastically increased, but upon the administration of 8-OHdG, it was found to be reduced (FIGS. 3a to 3d).

The pharmaceutical composition may further comprise a pharmaceutically acceptable carrier, excipient, or diluent, which is conventionally used in the preparation of a pharmaceutical composition, wherein the carrier can be a non-naturally occurring carrier. The carrier, excipient, or diluent may be lactose, dextrose, sucrose, sorbitol, mannitol, xylitol, erythritol, maltitol, starch, Acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oils.

Meanwhile, the pharmaceutical composition of the present invention may be formulated into oral preparations such as a powder, granule, tablet, capsule, suspension, emulsion, syrup, aerosol, etc., or formulated into a preparation for external use, suppository, or sterile solution for injection according to the conventional methods. When formulated into a preparation, a commonly used diluent or excipient such as a filler, an extender, a binder, a wetting agent, a disintegrating agent, a surfactant, etc. is used for preparation. Solid formulations for oral administration include a tablet, a pill, a powder, a granule, a capsule, etc., and are prepared by mixing at least one excipient, e.g., starch, calcium carbonate, sucrose, lactose, gelatin, etc., with the extract. Further, a lubricant such as magnesium stearate or talc may be included in addition to the excipient. Liquid preparations for oral administration include a suspension, a liquid for internal use, an emulsion, a syrup, etc., and also include various excipients such as a wetting agent, a sweetener, an aromatic, a preservative, etc., in addition to general simple diluents such as water and liquid paraffin. Preparations for parenteral administration include an aseptic aqueous solution, a non-aqueous formulation, a suspension, an emulsion, a freeze-dried formulation, and a suppository. As the non-aqueous formulation or suspension, propylene glycol; polyethylene glycol; vegetable oil such as olive oil; injectable ester such as ethyl oleate; etc. may be used. Witepsol, Macrogol, Tween 61, cocoa butter, laurin butter, glycerogelatin, etc. may be used as a base of the suppository.

The pharmaceutical composition may have any one formulation selected from the group consisting of a tablet, a pill, a powder, a granule, a capsule, a suspension, a liquid for internal use, an emulsion, a syrup, an aseptic aqueous solution, a non-aqueous solvent, a suspension, an emulsion, a lyophilized agent, and a suppository.

The amount of the 8-OHdG contained in the pharmaceutical composition of the present invention is not particularly limited, but may be 0.0001 wt % to 50 wt % based on the total weight of the final composition, and preferably 0.001 wt % to 10 wt %.

The pharmaceutical composition of the present invention may be administered in a pharmaceutically effective amount. As used herein, the term “pharmaceutically effective amount” refers to an amount sufficient for the treatment of diseases at a reasonable benefit/risk ratio applicable to a medical treatment, and the level of the effective dose may be determined from factors including severity of illness, drug activity, age, body weight, health conditions, drug sensitivity of a subject, administration time, administration route and dissolution rate, length of treatment of the pharmaceutical composition of the present invention, drug(s) used in combination with or simultaneously with the pharmaceutical composition of the present disclosure, and other factors well known in the medical field. The pharmaceutical composition of the present invention may be administered as an individual drug or in combination with other drug(s), and also sequentially or simultaneously with the conventional drug(s). Additionally, the pharmaceutical composition of the present invention may be administered as a single dose or in multiple divided doses. It is important that the minimum amount which can achieve the maximum effect without any side effects be administered in consideration of all of the factors described above.

The dose of the pharmaceutical composition of the present invention may be determined by a skilled person in the art considering the intended use(s), addiction level of a disease, age, body weight, sex, and anamnesis of a patient, or type of a substance used as an active ingredient, etc. For example, the pharmaceutical composition of the present invention may be administered in an amount of about 0.1 ng/kg to about 100 mg/kg for an adult, preferably from 1 ng/kg to 10 mg/kg. The pharmaceutical composition of the present invention may be administered once daily or in a few divided doses, but is not particularly limited thereto.

As another aspect, the present invention provides a method for preventing or treating liver fibrosis or liver cirrhosis, comprising administering the pharmaceutical composition to a subject having or at risk of developing liver fibrosis.

As used herein, the term “subject” includes animals such as horses, sheep, pigs, goats, camels, antelopes, dogs, etc. and humans, who are at risk of developing or have liver fibrosis or liver cirrhosis. Liver fibrosis or liver cirrhosis can be effectively prevented or treated by administering the pharmaceutical composition according to the present invention to a subject.

As used herein, the term “administration” refers to the introduction of a particular substance into a subject by an appropriate method. The pharmaceutical composition of the present invention may be administered via various oral and parenteral routes as long as it reaches a target tissue.

The pharmaceutical composition may be appropriately administered to a subject according to a method, route of administration, and dose conventionally employed in the art depending on the purpose or necessity. Examples of the administration routes may include oral, parenteral, subcutaneous, intraperitoneal, intrapulmonary, and intranasal administrations, and the parenteral administration may include intramuscular, intravenous, intraarterial, intraperitoneal, and subcutaneous administrations. Additionally, an appropriate dose and frequency of administration may be selected according to a method known in the art. The dose and the frequency of administration of the pharmaceutical composition of the present invention actually administered may be appropriately determined by various factors such as the type of the symptom to be treated, administration route, gender, physical conditions, diet, age and weight of a subject, and severity of the disease.

As used herein, the term “pharmaceutically effective amount” refers to an amount sufficient for the inhibition or alleviation of vasopermeability enhancement at a reasonable benefit/risk ratio applicable to a medical treatment, and the level of the effective dose may be determined from factors including severity of illness, drug activity, age, body weight, health conditions, drug sensitivity of a subject, administration time, administration route and dissolution rate, length of treatment of the pharmaceutical composition of the present invention, drug(s) used in combination with or simultaneously with the pharmaceutical composition of the present disclosure, and other factors well known in the medical field. The pharmaceutical composition of the present invention may be administered as an individual drug or in combination with other drug(s), and also sequentially or simultaneously with the conventional drug(s). Additionally, the pharmaceutical composition of the present invention may be administered as a single dose or in multiple divided doses. It is important that the minimum amount which can achieve the maximum effect without any side effects be administered in consideration of all of the factors described above.

As another aspect, the present invention provides a food composition for ameliorating liver fibrosis or liver cirrhosis, comprising 8-hydroxydeoxyguanosine (8-OHdG) or a pharmaceutically acceptable salt thereof.

The 8-OHdG provided in the present invention, as a DNA oxidation product, can be commonly recognized as a food product, and thus, it can be prepared in the form of a food composition to be used in the manufacture of a functional food product for alleviating liver fibrosis or liver cirrhosis.

The amount of the 8-OHdG or pharmaceutically acceptable salt thereof contained in the food composition is not particularly limited, but may be 0.0001 wt % to 10 wt %, more preferably 0.001 wt % to 1 wt % based on the total weight of the food composition. When the food is a drink, the amount may be 1 g to 10 g, preferably 2 g to 7 g, per 100 mL. When preparing a food, the composition can further include a conventionally used additional ingredient that may enhance smell, taste, appearance, etc. For example, vitamins A, C, D, E, B1, B2, B6, B12, niacin, biotin, folate, panthotenic acid, etc. as well as minerals such as zinc (Zn), iron (Fe), calcium (Ca), chromium (Cr), magnesium (Mg), manganese (Mn), copper (Cu), etc. may be included. Additionally, amino acids such as lysine, tryptophan, cysteine, valine, etc. can be included. Food additives such as preservatives (potassium sorbate, sodium benzoate, salicylic acid, sodium dehydroacetate, etc.), disinfectants (bleaching powder, higher bleaching powder, sodium hypochlorite, etc.), antioxidants (butylhydroxyanisole (BHA), butylhydroxytoluene (BHT), etc.), coloring agents (tar color, etc.), color-developing agents (sodium nitrite, etc.), bleaching agents (sodium sulfite), seasonings (monosodium glutamate (MSG), etc.), sweeteners (dulcin, cyclamate, saccharin, sodium, etc.), flavors (vanillin, lactones, etc.), swelling agents (alum, potassium hydrogen D-tartrate, etc.), fortifiers, emulsifiers, thickeners (adhesive pastes), film-forming agents, gum base agents, antifoaming agents, solvents, improvers, etc. may also be included. The food additives may be elected according to the type of the food and used in an appropriate amount.

Meanwhile, a functional food for ameliorating liver fibrosis or liver cirrhosis using the food composition for ameliorating liver fibrosis or liver cirrhosis, comprising 8-hydroxydeoxyguanosine (8-OHdG) or a pharmaceutically acceptable salt thereof.

As a specific example, a processed food for ameliorating liver fibrosis or liver cirrhosis may be prepared using the food composition. For example, a functional food may be prepared in the form of confectioneries, beverages, alcoholic beverages, fermented foods, canned foods, processed dairy foods, processed meat foods, or processed noodle foods. In particular, confectioneries may include biscuits, pies, cakes, breads, candies, jellies, gums, cereals (meal substitutes such as grain flakes, etc.), etc. Examples of beverages may include drinking water, carbonated drinks, functional ion drinks, juices (e.g., apple, pear, grape, aloe, tangerine, peach, carrot, tomato juices, etc.), sweet rice drinks, etc. Examples of alcoholic beverages may include refined rice wine, whiskey, soju, beer, liquor, fruit wine, etc. Examples of fermented foods may include soy sauce, soybean paste, red pepper paste, etc. Examples of canned foods may include canned marine products (e.g., canned products of tuna, mackerel, pacific saury, conch, etc.), canned meat products (canned products of beef, pork, chicken, turkey, etc.), canned agricultural products (canned products of corn, peach, pineapple, etc.), etc. Examples of processed dairy products may include cheese, butter, yogurt, etc. Examples of processed meat foods may include pork cutlet, beef cutlet, chicken cutlet, sausage, sweet-and-sour pork, nuggets, Neobiani, etc. Noodles such as sealing-packaged wet noodles may be included. Additionally, the food composition may be used in retort foods, soups, etc.

As used herein, the term “functional food”, also called “food for special health use (FoSHU)”, refers to a food with high medicinal and medical effects which efficiently exhibits a bioregulatory function in addition to a function of nutrient supply. The functional food may be prepared in various forms such as tablets, capsules, powders, granules, liquids, pills, etc., to obtain useful effects for ameliorating liver fibrosis or liver cirrhosis.

As still another aspect, the present invention provides use of 8-hydroxydeoxyguanosine (8-OHdG) or a pharmaceutically acceptable salt thereof for the prevention or treatment of liver fibrosis or liver cirrhosis.

### ADVANTAGEOUS EFFECTS

Use of the pharmaceutical composition for preventing or treating liver fibrosis or liver cirrhosis can serve to reduce the levels of various biomarkers whose expression levels increase due to fibrosis. Accordingly, the pharmaceutical composition can be widely used in the prevention or treatment of liver fibrosis or liver cirrhosis induced thereby.

### BRIEF DESCRIPTION OF DRAWINGS

FIG. 1 is a graph showing the effect of 8-OHdG on the level of hydroxyproline measured in the liver fibrosis animal model.

FIG. 2a is a graph showing the effect of 8-OHdG on the level of mRNA of TGF-β measured in the liver fibrosis animal model.

FIG. 2b is a graph showing the effect of 8-OHdG on the level of mRNA of TIMP-1 measured in the liver fibrosis animal model.

FIG. 2c is a graph showing the effect of 8-OHdG on the level of mRNA of collagen measured in the liver fibrosis animal model.

FIG. 2d is a graph showing the effect of 8-OHdG on the level of mRNA of α-SMA measured in the liver fibrosis animal model.

FIG. 2e is a graph showing the effect of 8-OHdG on the level of mRNA of NOX1 measured in the liver fibrosis animal model.

FIG. 2f is a graph showing the effect of 8-OHdG on the level of mRNA of NOX2 measured in the liver fibrosis animal model.

FIG. 3a is a graph showing the effect of 8-OHdG on the level of protein of collagen measured in the liver fibrosis animal model.

FIG. 3b is a photo of western blot results showing the effect of 8-OHdG on the level of protein of α-SMA measured in the liver fibrosis animal model.

FIG. 3c is a photo showing the effect of 8-OHdG on the level of protein of immunostained NOX1, measured in the liver fibrosis animal model.

FIG. 3d is a photo showing the effect of 8-OHdG on the level of protein of immunostained NOX2, measured in the liver fibrosis animal model.

### DETAILED DESCRIPTION OF THE INVENTION

Hereinafter, the present invention will be described in details with reference to the following Examples. However, these Examples are for illustrative purposes only, and the scope of the present invention is not limited to these Examples.

### Example 1: Preparation of Sample

250 g to 300 g male Sprague-Dawley rats were subject to bile duct ligation (BDL) and atresia of bile duct to prepare liver fibrosis animal models.

The prepared animal models were used to prepare one control group and two experimental groups.

Seven of the animal models were laparotomized, ligated, and recovered to be used for the control group.

Six of the animal models were laparotomized, and the proximal and distal bile ducts were ligated and then cut in the middle to separate the bile duct. The animal models were then sutured and allowed to recover to be used for Experimental Group 1.

Six animal models treated in the same manner as experimental group 1 were sutured and administered with 8-OHdG in the amount of 60 mg/kg/day for 3 weeks to be used for Experimental Group 2.

The animal models of each of the control and experimental groups were sutured and raised for 3 weeks, followed by taking blood and liver tissues therefrom to be used for subsequent experimental procedures.

### Example 2: Effect of 8-OHdG on the Level of Hydroxyproline in Liver Tissue

The effect of 8-OHdG on the level of hydroxyproline, known as a biomarker of liver fibrosis, in the liver tissue was to be verified.

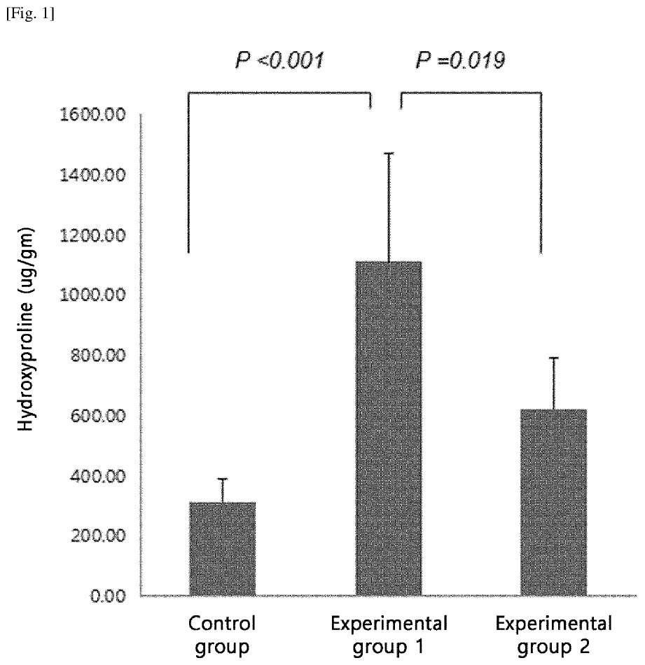

Specifically, 100 μL of distilled water was added to 10 mg of liver tissues of the control and experimental groups prepared in Example 1 and homogenized, and 100 μL of 12 N HCL was added to 100 μL of the homogenized sample and allowed to react for 3 hours at 120° C. Upon termination of the reaction, 10 μL of the reaction solution was placed in each well of a 96-well plate and dried. After adding 100 μL of chloramine T, the reaction solution was reacted for 90 minutes at 60° C. After the reaction, absorbance at 560 nm was measured to calculate the level of hydroxyproline in the liver tissue and compared (FIG. 1).

FIG. 1 is a graph showing the effect of 8-OHdG on the level of hydroxyproline measured in the liver fibrosis animal model. As shown in the figure, the hydroxyproline level in the liver tissue of the liver fibrosis animal model was remarkably increased (Experimental Group 1), whereas the increased level of hydroxyproline was reduced by 8-OHdG treatment (Experimental Group 2).

### Example 3: Effect of 8-OHdG on the mRNA Level of the Liver Fibrosis Biomarkers in Liver Tissue

The effect of 8-OHdG on the mRNA level of TGF-β,l TIMP-1, collagen, α-SMA, NOX1, and NOX2, known biomarkers of liver fibrosis and ROS formation process, in liver tissues were to be verified.

### Example 3-1: Change of TGF-β on mRNA Level

Total RNA was extracted from the control and experimental groups prepared in Example 1 using RNeasy Mini Kit (Qiagen, Hilden, Germany), and the extracted total RNA was reverse translated using high-capacity cDNA reverse transcription kit (Applied Biosystems, Foster City, Calif.) to obtain cDNA.

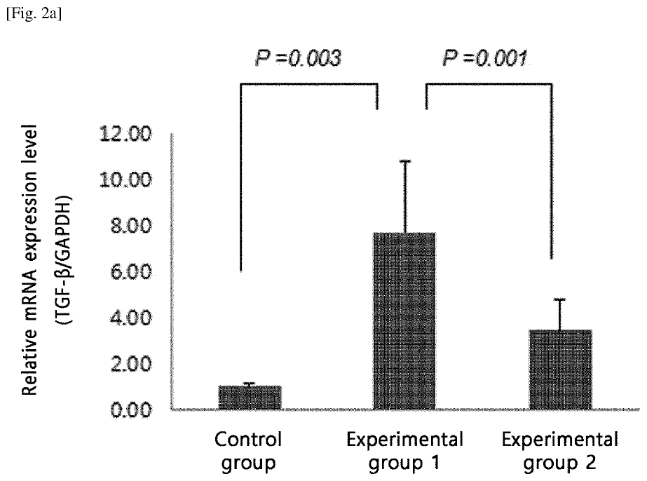

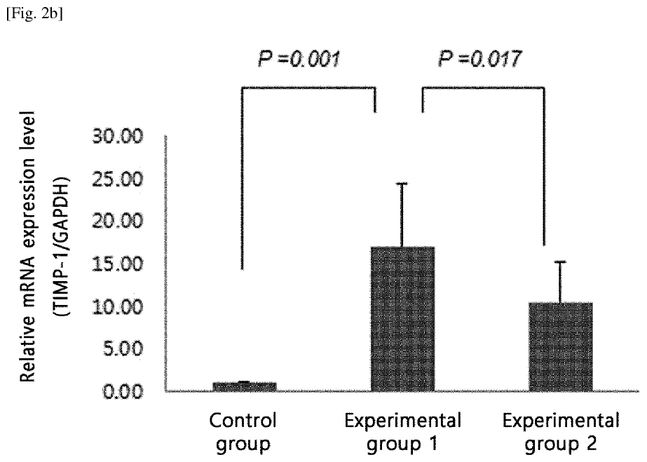

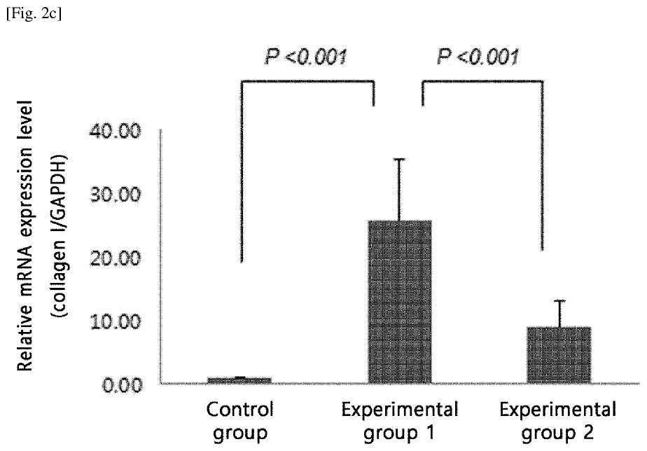

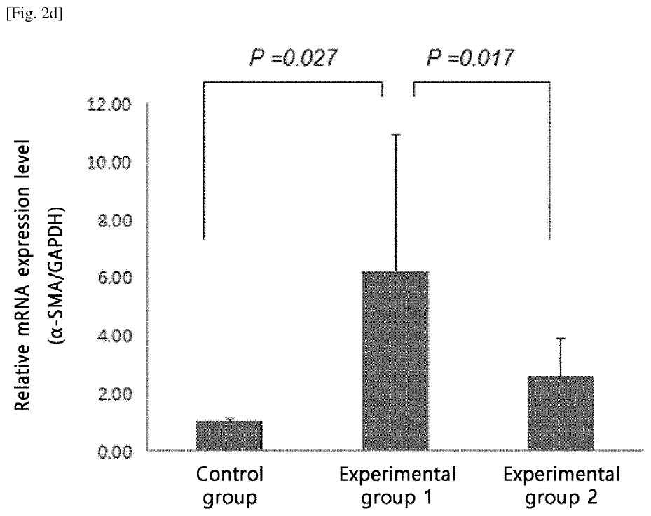

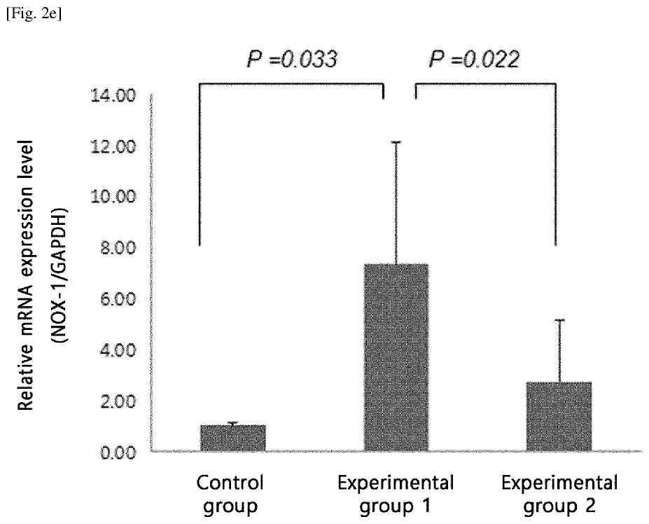

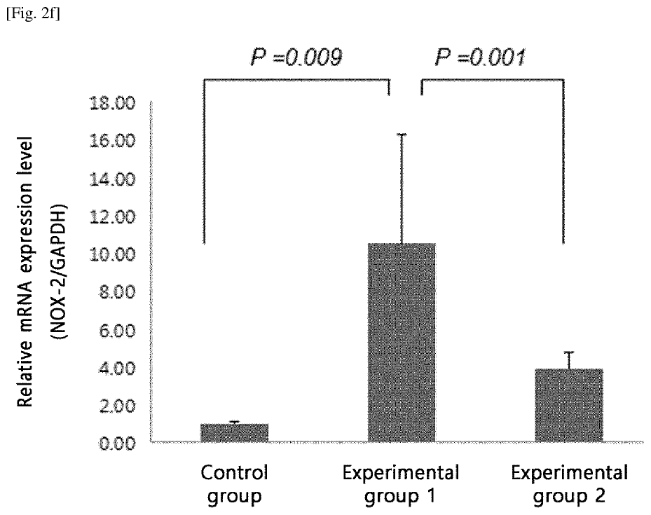

qPCR was performed using thus-obtained cDNA as a template, as well as the primers (below), the Bio-Rad CFX96 real-time PCR detection system (Bio-Rad, Hercules, Calif.), and an SYBR Premix Ex Taq II kit (Takara Biotechnology) to measure and compare the mRNA levels of TGF-β in the control and experimental groups (FIG. 2a). The mRNA of GAPDH was used as an internal control.

(SEQ ID NO: 1)
TGF-β F: 5′-AGAAGTCACCCGCGTGCTAA-3′
(SEQ ID NO: 2)
TGF-β R: 5′-TCCCGAATGTCTGACGTATTGA-3′

FIG. 2a is a graph showing the effect of 8-OHdG on the level of mRNA of TGF-β measured in the liver fibrosis animal model. As shown in the figure, the mRNA level of TGF-β in the liver tissue of the liver fibrosis animal model was remarkably increased (Experimental Group 1), whereas the increased level of mRNA was reduced by 8-OHdG treatment (Experimental Group 2).

### Example 3-2: Change of TIMP-1 on mRNA Level

Except that the primers below were used instead, the same method was performed as in Example 3-1 to measure and compare the mRNA levels of TIMP-1 in the liver tissues of the control and experimental groups (FIG. 2b).

(SEQ ID NO: 3)
TIMP-1 F: 5′-TCCTCTTGTTGCTATCATTGATAGCTT-3′
(SEQ ID NO: 4)
TIMP-1 R: 5′-CGCTGGTATAAGGTGGTCTCGAT-3′

FIG. 2b is a graph showing the effect of 8-OHdG on the level of mRNA of TIMP-1 measured in the liver fibrosis animal model. As shown in the figure, the mRNA level of TIMP-1 in the liver tissue of the liver fibrosis animal model was remarkably increased (Experimental Group 1), whereas the increased mRNA level of TIMP-1 was reduced by 8-OHdG treatment (Experimental Group 2).

### Example 3-3: Changeof Collagen on mRNA Level

Except that the primers below were used instead, the same method was performed as in Example 3-1 to measure and compare the mRNA levels of collagen in the liver tissues of the control and experimental groups (FIG. 2c).

(SEQ ID NO: 5)
Collagen Iα1 F: 5′-TGCCGATGTCGCTATCCA-3′
(SEQ ID NO: 6)
Collagen Iα1 R: 5′-TCTTGCAGTGATAGGTGATGTTCTG-3′

FIG. 2c is a graph showing the effect of 8-OHdG on the level of mRNA of collagen measured in the liver fibrosis animal model. As shown in the figure, the mRNA level of collagen in the liver tissue of the liver fibrosis animal model was remarkably increased (Experimental Group 1), whereas the increased mRNA level of collagen was reduced by 8-OHdG treatment (Experimental Group 2).

### Example 3-4: Change of α-SMA on mRNA Level

Except that the primers below were used instead, the same method was performed as in Example 3-1 to measure and compare the mRNA levels of α-SMA in the liver tissues of the control and experimental groups (FIG. 2d).

(SEQ ID NO: 7)
α-SMA F: 5′-GCTGACAGGATGCAGAAGGA-3′
(SEQ ID NO: 8)
α-SMA R: 5′-GCCGATCCAGACAGAATATTTG-3′

FIG. 2d is a graph showing the effect of 8-OHdG on the level of mRNA of α-SMA measured in the liver fibrosis animal model. As shown in the figure, the mRNA level of α-SMA in the liver tissue of the liver fibrosis animal model was remarkably increased (Experimental Group 1), whereas the increased mRNA level of α-SMA was reduced by 8-OHdG treatment (Experimental Group 2).

### Example 3-5: Change of NOX1 on mRNA Level

Except that the primers below were used instead, the same method was performed as in Example 3-1 to measure and compare the mRNA levels of NOX1 in the liver tissues of the control and experimental groups (FIG. 2e).

(SEQ ID NO: 9)
NOX1 F: 5′-CTACAGTAGAAGCCAACAGGCCAT-3′
(SEQ ID NO: 10)
NOX1 R: 5′-ACTGTCACGTTTGGAGACTGGATG-3′

FIG. 2e is a graph showing the effect of 8-OHdG on the level of mRNA of NOX1 measured in the liver fibrosis animal model. As shown in the figure, the mRNA level of a-SMA in the liver tissue of the liver fibrosis animal model was remarkably increased (Experimental Group 1), whereas the increased mRNA level of NOX1 was reduced by 8-OHdG treatment (Experimental Group 2).

### Example 3-6: Change of NOX2 on mRNA Level

Except that the primers below were used instead, the same method was performed as in Example 3-1 to measure and compare the mRNA levels of NOX2 in the liver tissues of the control and experimental groups (FIG. 2f).

(SEQ ID NO: 11)
NOX2 F: 5′-CTACAGTAGAAGCCAACAGGCCAT-3′
(SEQ ID NO: 12)
NOX2 R: 5′-ACTGTCACGTTTGGAGACTGGATG-3′

FIG. 2f is a graph showing the effect of 8-OHdG on the level of mRNA of NOX2 measured in the liver fibrosis animal model. As shown in the figure, the mRNA level of α-SMA in the liver tissue of the liver fibrosis animal model was remarkably increased (Experimental Group 1), whereas the increased mRNA level of NOX2 was reduced by 8-OHdG treatment (Experimental Group 2).

In light of the experimental results of Examples 3-1 to 3-6, the mRNA levels of TGF-β, TIMP-1, collagen, α-SMA, NOX1, and NOX2, known biomarkers of liver fibrosis and ROS formation process, increased in the liver tissues of the liver fibrosis animal models; however, upon administration with 8-OHdG, the increased mRNA levels of the biomarkers were reduced.

### Example 4: Effect of 8-OHdG on the Level of Protein of Liver Fibrosis Marker in the Liver Tissue

From the result of Example 3, the mRNA levels of TGF-β, TIMP-1, collagen, α-SMA, NOX1, and NOX2, known biomarkers of liver fibrosis and ROS formation process, in the liver tissues were confirmed to be reduced by administration of 8-OHdG. In this regard, whether the same effect would be exhibited in terms of protein was to be verified.

### Example 4-1: Change of Collagen on Protein Level

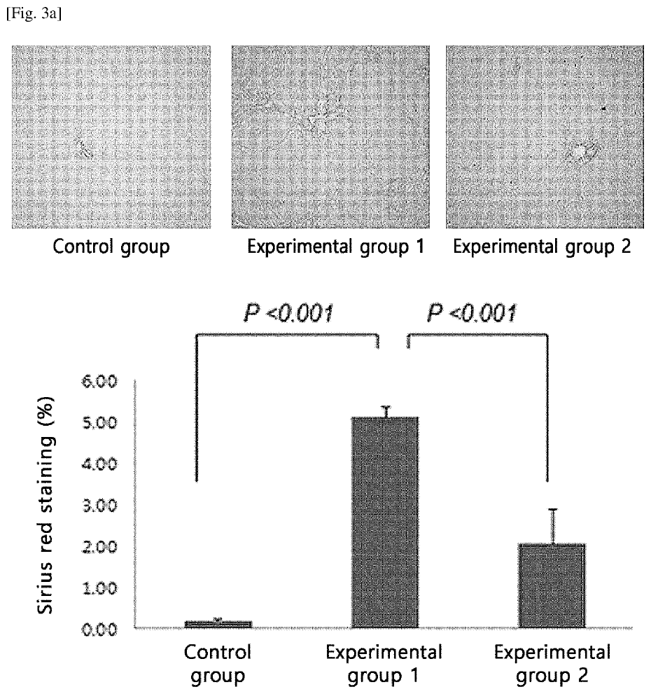

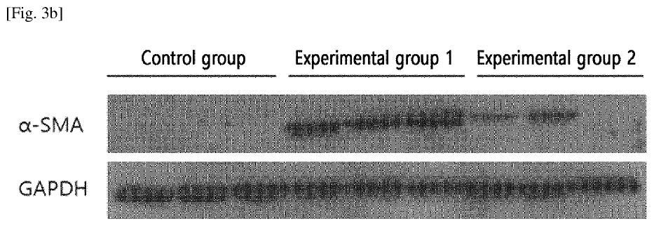

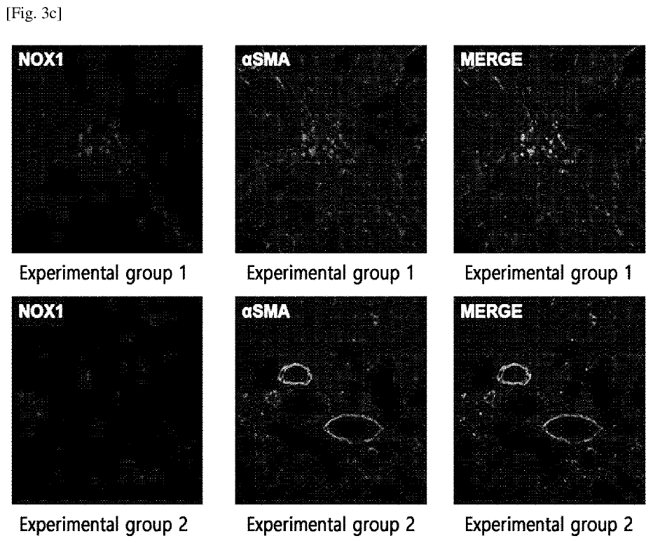

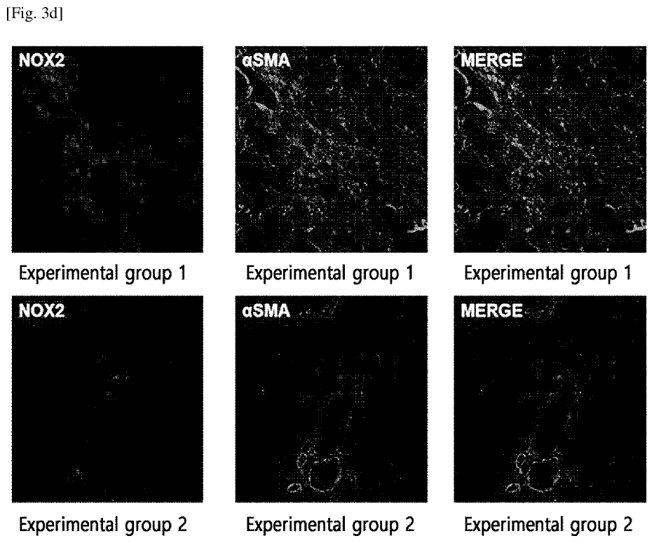

Collagen of the liver tissues of the control and experimental groups prepared in Example 1 was stained with Picro Sirius Red Stain kit (Abcam Company Ltd. China), and the level of the collagen labeled in red was analyzed using ImageJ software (NIH, USA) (FIG. 3a).

FIG. 3a is a microscopic image and a graph showing the effect of 8-OHdG on the level of protein of immunostained NOX1, measured in the liver fibrosis animal model. As shown in the figure, the protein level of collagen in the liver tissue of the liver fibrosis animal model was remarkably increased (Experimental Group 1), whereas the increased protein level of collagen was reduced by 8-OHdG treatment (Experimental Group 2).

### Example 4-2: Change of α-SMA on Protein Level

The protein level of α-SMA, expressed in the liver tissues of the control and experimental groups prepared in Example 1, was measured by western blot using anti-α-SMA antibodies (FIG. 3b). GAPDH protein was used as an internal control.

FIG. 3b is a photo of western blot results showing the effect of 8-OHdG on the level of protein of α-SMA measured in the liver fibrosis animal mode. As shown in the figure, the protein level of α-SMA in the liver tissue of the liver fibrosis animal model was remarkably increased (Experimental Group 1), whereas the increased protein level of α-SMA was reduced by 8-OHdG treatment (Experimental Group 2).

### Example 4-3: Change of NOX1 on Protein Level

Immunofluorescent staining of NOX1 was performed on the liver tissues of the control and experimental groups prepared in Example 1 using anti-NOX1 antibodies, secondary antibody conjugated with Alexa Fluor 594, and secondary antibody conjugated with Alexa Fluor 488, and the results were compared (FIG. 3c). α-SMA was used as a hepatic stellate cell activity index.

FIG. 3c is a photo showing the effect of 8-OHdG on the level of protein of immunostained NOX1, measured in the liver fibrosis animal model. As shown in the figure, the protein level of NOX1 in the liver tissue of the liver fibrosis animal model was remarkably increased (Experimental Group 1), as was that of α-SMA, used as a hepatic stellate cell activity index, whereas the increased protein levels of α-SMA and NOX1 were reduced by 8-OHdG treatment (Experimental Group 2).

### Example 4-4: Change of NOX2 on Protein Level

Immunofluorescent staining of NOX2 was performed on the liver tissues of the control and experimental groups prepared in Example 1 using anti-NOX2 antibodies, secondary antibody conjugated with Alexa Fluor 594, and secondary antibody conjugated with Alexa Fluor 488, and the results were compared (FIG. 3d). α-SMA was used as a hepatic stellate cell activity index.

FIG. 3d is a photo showing the effect of 8-OHdG on the level of protein of immunostained NOX2, measured in the liver fibrosis animal model. As shown in the figure, the protein level of NOX2 in the liver tissue of the liver fibrosis animal model was remarkably increased (Experimental Group 1), as was that of α-SMA used as a hepatic stellate cell activity index, whereas the increased protein levels of α-SMA and NOX2 were reduced by 8-OHdG treatment (Experimental Group 2).

In light of the results of Examples 4-1 to 4-4, the protein levels of TGF-β, TIMP-1, collagen, α-SMA, NOX1, and NOX2, known biomarkers of liver fibrosis and ROS formation process, increased in the liver tissues of the liver fibrosis animal models; however, upon administration with 8-OHdG, the increased protein levels of the biomarkers were reduced.

In conclusion, the levels of the biomarkers of liver fibrosis and ROS formation process, which were increased due to induced liver fibrosis, are reduced by administration of 8-OHdG in liver fibrosis animal model. Accordingly, the 8-OHdG can be used for the treatment of liver fibrosis.

From the foregoing, a skilled person in the art to which the present disclosure pertains will be able to understand that the present disclosure may be embodied in other specific forms without modifying the technical concepts or essential characteristics of the present disclosure. In this regard, the exemplary embodiments disclosed herein are only for illustrative purposes and should not be construed as limiting the scope of the present disclosure. On the contrary, the present disclosure is intended to cover not only the exemplary embodiments but also various alternatives, modifications, equivalents, and other embodiments that may be included within the spirit and scope of the present disclosure as defined by the appended claims.

1. A pharmaceutical composition for preventing or treating liver fibrosis, comprising 8-hydroxydeoxyguanosine (8-OHdG) or a pharmaceutically acceptable salt thereof.

2. The pharmaceutical composition of claim 1, wherein the 8-OHdG inhibits expression of a liver fibrosis marker selected from the group consisting of hydroxyproline, transforming growth factor β (TGF-β), tissue inhibitor of metalloproteinase 1 (TIMP-1), collagen, α-smooth muscle actin (α-SMA), NADPH oxidase 1 (NOX1), NADPH oxidase 2 (NOX2), ras-related C3 botulinum toxin substrate 1 (Rac1), and a combination thereof in a liver tissue.

3. The pharmaceutical composition of claim 1, further comprising a pharmaceutically acceptable carrier, excipient, or diluent.

4. A method for preventing or treating liver fibrosis or liver cirrhosis, comprising administering the pharmaceutical composition of any one of claims 1 to 3 to a subject having or at risk of developing liver fibrosis.

5. A food composition for ameliorating liver fibrosis or liver cirrhosis, comprising 8-hydroxydeoxyguanosine (8-OHdG) or a pharmaceutically acceptable salt thereof.


SEQUENCE LISTING (12 sequences)

SEQ ID NO: 1 - DNA, 20 bp, Artificial Sequence
  primer
agaagtcacccgcgtgctaa

SEQ ID NO: 2 - DNA, 22 bp, Artificial Sequence
  primer
tcccgaatgtctgacgtattga

SEQ ID NO: 3 - DNA, 27 bp, Artificial Sequence
  primer
tcctcttgttgctatcattgatagctt

SEQ ID NO: 4 - DNA, 23 bp, Artificial Sequence
  primer
cgctggtataaggtggtctcgat

SEQ ID NO: 5 - DNA, 18 bp, Artificial Sequence
  primer
tgccgatgtcgctatcca

SEQ ID NO: 6 - DNA, 25 bp, Artificial Sequence
  primer
tcttgcagtgataggtgatgttctg

SEQ ID NO: 7 - DNA, 20 bp, Artificial Sequence
  primer
gctgacaggatgcagaagga

SEQ ID NO: 8 - DNA, 22 bp, Artificial Sequence
  primer
gccgatccagacagaatatttg

SEQ ID NO: 9 - DNA, 24 bp, Artificial Sequence
  primer
ctacagtagaagccaacaggccat

SEQ ID NO: 10 - DNA, 24 bp, Artificial Sequence
  primer
actgtcacgtttggagactggatg

SEQ ID NO: 11 - DNA, 24 bp, Artificial Sequence
  primer
ctacagtagaagccaacaggccat

SEQ ID NO: 12 - DNA, 24 bp, Artificial Sequence
  primer
actgtcacgtttggagactggatg